(19) United States
(12) Patent Application Publication
(10) Pub. No.: US 2012/0172781 A1
(43) Pub. Date: Jul. 5, 2012

(54) METHOD OF RESUSCITATION

(76) Inventor: Yanming Wang, Malden, MA (US)

(21) Appl. No.: 12/983,379

(22) Filed: Jan. 3, 2011

(51) Int. Cl. A61M 25/10; A61M 1/36; A61M 5/14

(52) U.S. Cl. 604/6.16; 604/509; 604/506

(57) ABSTRACT

This disclosure presents a method and apparatus to perfuse heart and/or other organs with a resuscitation fluid to replace blood circulation in the vascular system to resuscitate cardiac arrest patient.

### FIELD OF THE DISCLOSURE

This disclosure relates to the field of emergency medicine. In particular, the present disclosure relates to a method and apparatus of perfusing a resuscitation fluid in the vascular system to treat cardiac arrest patients.

### BACKGROUND

Blood circulation sustains life by carrying oxygen and nutrients to all the cells in the body, and carries away carbon dioxide and waste products so that they can be removed from the body. The heart is the pump of blood circulation system. It pumps blood throughout the blood vessels by repeated, rhythmic contractions. Without access to oxygen and nutrients, cells and body tissues die.

Cardiac arrest is the cessation of circulation of the blood due to failure of the heart to contract effectively. A cardiac arrest is usually diagnosed clinically by the absence of a pulse. When this happens, the heart abruptly losses pumping function, oxygen and nutrients cannot be delivered to the cells in the body, and carbon dioxide and waste products cannot be removed away. Vital organs, such as heart, brain have limited tolerance to cardiac arrest. For examples, it is estimated that 5-8 minutes of cardiac arrest is known to result in severe brain damage. Therefore, it is important to provide oxygen and other nutrients to the whole body while trying to achieve cardiac Return of Spontaneous Circulation (ROSC; i.e., the heart starts to beat on its own again after cardiac arrest). Thus, cardiac arrest causes clinical death if it's not treated within minutes.

Cardiac arrest is one of the leading causes of death all over the world. It is estimated that more than 350,000 people died of sudden cardiac arrest each year in USA. In Europe, it affects about 700,000 individuals each year. In China, its annual incidence reaches about 41.8 of 100,000 populations. People who have heart disease are at increased risk for cardiac arrest. However, most cardiac arrest happens in people who appear healthy and have no known heart disease or other risk factors for cardiac arrest. The common causes of cardiac arrest usually include the following: 1. Coronary artery disease. It accounts for about 80% incidence of cardiac arrest. Many cases are the results of ventricular fibrillation (VF), a condition that ventricular muscle twitches randomly, rather than contracting in a coordinated fashion, so the ventricles fail to pump blood into the arteries and into systemic circulation. 2. Non-ischemic heart diseases, including cardiomyopathy, hypertensive heart disease, congestive heart failure, coronary artery abnormalities, myocarditis, and hypertrophic cardiomyopathy. 3. Non-cardiac causes such as, trauma, hemorrhagic shock (hypovolemia) or non-trauma related bleeding (such as gastrointestinal bleeding, aortic rupture, and intracranial hemorrhage), asphyxiation, drug overdose, intoxication, choking, drowning, electric shock, airway obstruction, pulmonary embolism, hypoxia, acidosis, hyperkalemia or hypokalemia (excess and inadequate potassium), hypothermia, hypoglycemia or hyperglycemia, intoxication, cardiac tamponade, tension pneumothorax, and bacterial and viral infection. 4. Risk factors such as smoking, severe physical stress, obesity, diabetes, and family history (such as long QT syndrome). 5. Cardioplegic arrest: Open heart surgery requires that heart beat to be stopped by cardioplegic solution. Aortic surgery requires that aorta to be cross-clamped causing systemic arrest blow the cross-clamped aorta.

A common treatment for cardiac arrest is known as cardiopulmonary resuscitation (CPR). The theoretical basis for contemporary CPR practice including ventilation, closed chest compressions, open chest cardiac massage and defibrillation was established in the 1960's. The fundamental strategies for CPR have not changed since these original concepts emerged. European Resuscitation Council and American Heart Association review and publish guidelines every five years reflecting the update for the CPR practice. Recent CPR guidelines can be found in “2005 American Heart Association Guidelines for Cardiopulmonary Resuscitation and Emergency Cardiovascular Care” Circulation. 2005:112:IV-1-IV-18 and “European Resuscitation Council Guidelines for Resuscitation 2010” Resuscitation 2010: 81:1219-1276). Despite numerous scientific advances throughout modern medicine, outcome of resuscitation for cardiac arrest victims remains poor. For witnessed in-hospital cardiac arrest, the ROSC is about 48% and the survival is 22%; for unwitnessed in-hospital cardiac arrest, the ROSC is about 21% and the survival is 1%; for Bystander CPR, the ROSC is about 40% and the survival is 4%; for No Bystander CPR (Ambulance CPR), the ROSC is about 15% and the survival is 2%; for Defibrillation within 3-5 minutes, the ROSC is about 74% and the survival is 30%. The ROSC represents one of the key factors for successful CPR. The sooner the ROSC is achieved, the higher the chances for a cardiac arrest patient to survive. Often times, the immediate CPR cannot be initiated until medical first responder is available. When the interval of cardiac arrest becomes longer, it becomes more difficult for conventional CPR to achieve the ROSC. Moreover, chest compressions often cause significant local blunt trauma, including bruising or fracture of the sternum or ribs.

### SUMMARY

All organs in the body depend on oxygen and nutrients for their viability. The blood circulation carries oxygen and nutrients to the body. Many physiological saline based fluid contain basic nutrients. These aqueous solutions can carry a certain amount of oxygen as well. The inventor discovered that, although the oxygen carrying capability of these solutions is much less than that of the blood, they can replace blood to provide enough oxygen and nutrients to keep organs viable in the body during emergency condition such as cardiac arrest. This disclosure presents a method of perfusing a physiological saline based fluid as the resuscitation fluid to replace blood circulation in the vascular system to deliver oxygen and nutrients to resuscitate a cardiac arrest patient. Resuscitation fluid perfusion to heart alone can reach ROSC. Resuscitation fluid perfusion to multiple organs or the whole body can keep all organs and cells viable during cardiac arrest. In addition to provide oxygen and nutrients, the resuscitation fluid also serves as an effective coolant for therapeutic hypothermia. The resuscitation fluid can be maintained at desired temperature according to the resuscitation need. Perfusion with a resuscitation fluid can adjust and maintain the body temperature at a desired temperature with ease. For example, moderate hypothermia (28° C. to 34° C.), severe hypothermia (13° C. to 28° C.), or even severest hypothermia (0.1° C. to 13° C.) can be achieved.

0.9% Sodium Chloride is isotonic, its oxygen partial pressure (PO₂) is at least 120 mmHg at 37° C. under normal atmosphere. So 0.9% Sodium Chloride can be used as a basic resuscitation fluid to sustain cellular life. Other nutrients, such as K⁺, Ca²⁺, P, Mg²⁺, HCO₃⁻, glucose, plasma protein (albumin) and insulin, and amino acids, vitamins etc, can be added into 0.9% Sodium Chloride for better resuscitation fluid with integrated nutrients. The Krebs-Henseleit solution, Tyrode solution, Ringer's solution, serum, cell culture media and their variants etc. can all be considered as the physiological saline based fluid.

A typical Krebs-Henseleit solution has the following composition: NaCl 118.5 mM, NaHCO₃ 25.0 mM, KCl 4.7 mM, MgSO₄ 1.2 mM, glucose 11 mM, and CaCl₂ 2.5 mM, have a pH of 7.4 at 37° C. when it is continuously gassed with 5% CO₂.

A typical Tyrode solution has the following composition: NaCl 128.3 mM, KCl 4.7 mM, CaCl₂ 1.36 mM, MgCI₂ 1.05 mM, NaHCO₃ 20.2 mM, NaH₂PO₄ 0.42 mM, and glucose 11.1 mM, and has a pH of 7.4 at 37° C. when it is continuously gassed with 5% CO₂.

Ringer's solution has the following composition: Na⁺ 130 mM, Cl⁻ 109 mM, K⁺ 4 mM, and Ca²⁺ 1.5 mM.

Serum is collected after blood coagulation. It contains all components of blood except blood cells and clotting factors.

Cell culture media mimicking serum to support growth of cells derived from animals. Examples of cell culture media include RPMI1640, MEM, DMEM, and Neurobasal etc. The culture media were described in many researchers (Eagle. Nutrition needs of mammalian cells in tissue culture. Science. 1955 Vol. 122, No 3168.501-504. Hanss & Moore. Studies of culture media for the growth of human tumor cells. Experimental cell research. 1964 34: 243-256). Although the composition can be very complicated, the basic nutrition of cell culture media usually contain inorganic salts, amino acids, vitamins, glucose, growth factors, insulin, and plasma proteins.

A preferred resuscitation fluid is an analog of serum as follow: Na⁺ 120-155 meq/L, Cl⁻ 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L, and water. The osmolality of the resuscitation fluid is typically between 280-500 mOsm/L. The pH of the resuscitation fluid is typically between 7-7.45. The PO₂ of the resuscitation fluid is generally above 100 mm Hg and the PCO₂ of the fluid is generally below 40 mm Hg. The resuscitation fluid can be made with right ranges of pH, PO₂ and PCO₂ and packed in a sealed container, which is not exposed to atmosphere during the perfusion. Optionally, the resuscitation fluid can be continuously gassed with O₂ and CO₂ during perfusion. An example of the resuscitation fluid can include the following ingredients: Na⁺ 130 meq/L, Cl⁻ 140 meq/L, K⁺ 3.5 meq/L, Ca²⁺ 2.5 meq/L, Mg²⁺ 2 meq/L, HCO₃⁻ 25 meq/L, Glucose 100 mg/dl, albumin 0.1 gram/dl, insulin 10 IU/L, heparin 10 U/L, and water. The pH of this resuscitation fluid can be about 7.4 when it is continuously oxygenated with gas mixture of CO₂ and air or O₂. The preferred proportion of gas mixture is 5% of CO₂: 95% of air or 95% of O₂.

Other preferred resuscitation fluids are cell culture media include the following ingredients: Na⁺ 120-155 meq/L, Cl⁻ 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L, L-Arginine.HCl 0-0.2 g/L, L-Cystine.2HCl 0-0.2 g/L, L-Glutamine 0-0.2 g/L, Glycine 0-0.2 g/L, L-Histidine.HCl.H₂O 0-0.2 g/L, L-Isoleucine 0-0.2 g/L, L-Leucine 0-0.2 g/L, L-Lysine.HCl 0-0.2 g/L, L-Methionine 0-0.2 g/L, L-Phenylalanine 0-0.2 g/L, L-Serine 0-0.2 g/L, L-Threonine 0-0.2 g/L, L-Tryptophan 0-0.2 g/L, L-Tyrosine.2Na.2H₂O 0-0.2 g/L, L-Valine 0-0.2 g/L, Choline Chloride 0-0.01 g/L, Folic Acid 0-0.01 g/L, myo-Inositol 0-0.01 g/L, Nicotinamide 0-0.01 g/L, D-Pantothenic Acid 0-0.01 g/L, Pyridoxine.HCl 0-0.01 g/L, Riboflavin 0-0.001 g/L, Thiamine.HCl 0-0.01 g/L, Vitamin B-12 0-0.001 g/L. The pH of the resuscitation fluids can be about 7-7.45 when it is continuously oxygenated with gas mixture of CO₂ and air or O₂. The preferred proportion of gas mixture is 5% of CO₂ and 95% of air or 5% of CO₂ and 95% of O₂. The cell culture medium is most preferred for long term perfusion, e.g. perfusion for several days to several months. An example is the DMEM cell culture medium which include the following: NaCl 6.4 g/L CaCl₂ 0.2 g/L, MgSO₄ 0.0977 g/L, KCl 0.4/L, NaHCO₃ 1.5 g/L, NaH₂PO₄.H₂O 0.125 g/L, L-Arginine.HCl 0.084 g/L, L-Cystine.2HCl 0.0626 g/L, L-Glutamine 0.584 g/L, Glycine 0.03 g/L, L-Histidine.HCl.H₂O 0.042 g/L, L-Isoleucine 0.105 g/L, L-Leucine 0.105 g/L, L-Lysine.HCl 0.146 g/L, L-Methionine 0.03 g/L, L-Phenylalanine 0.066 g/L, L-Serine 0.042 g/L, L-Threonine 0.095 g/L, L-Tryptophan 0.016 g/L, L-Tyrosine.2Na.2H₂O 0.10379 g/L, L-Valine 0.094 g/L, Choline Chloride 0.004 g/L, Folic Acid 0.004 g/L, myo-Inositol 0.0072 g/L, Nicotinamide 0.004 g/L, D-Pantothenic Acid 0.004 g/L, Pyridoxine.HCl 0.004 g/L, Riboflavin 0.0004 g/L, Thiamine.HCl 0.004 g/L, Vitamin B-12 0.00068 g/L, D-Glucose 1 g/L, Insulin 4 mg/L, and Albumin 2.5 g/L).

When resuscitation can be started soon after cardiac arrest, resuscitation fluid can be perfused exclusively to the heart, for example, within 10-30 minutes or even hours if ambient temperature is low. Under this circumstance, quick establishment of ROSC is generally the main goal because cardiac arrest allows a short period of therapeutic window, during which the vital organ damage is reversible. The inventor discovered that resuscitation fluid perfusion of the heart can achieve this goal. This can be accomplished by creating an isolated environment to solely perfuse the heart with a resuscitation fluid via three balloon catheters. For example, a first catheter having a balloon and a lumen can be introduced from a peripheral artery into the aorta (preferably, the proximal end of the lumen to ascending aorta). The balloon can then be inflated to occlude the aorta, the distal end of the lumen can be connected to a first external reservoir where resuscitation fluid is oxygenated continuously with gas mixture of CO₂ and air or O₂. The preferred proportion of gas mixture is 5% of CO₂ and 95% of atmospheric air or 5% of CO₂ and 95% of O₂. The O₂ can dissolve in the resuscitation fluid when the fluid is exposed to the atmospheric air or 95% O₂. The PO₂ of the resuscitation fluid can reach 154-158 mmHg if it is exposed to normal atmosphere air at sea level. It is calculated that this can provide O₂ of 0.45 ml/100 ml of resuscitation fluid. The resuscitation fluid PO₂ can reach 500-600 mmHg if it is exposed to the 95% O₂. It is calculated that this can provide O₂ of 1.5 ml/100 ml of resuscitation fluid. The 5% CO₂ is typically used for keeping pH at physiological level. The fluid communication can be established between the first external reservoir and the heart via the first catheter. Optionally, the first external reservoir can be replaced by a bubble or membrane oxygenator and blood reservoir used in cardiopulmonary bypass surgery to oxygenate resuscitation fluid. A second and a third catheter having a balloon and a lumen can be introduced from peripheral veins into right heart via superior vena cava and inferior vena cava (preferably, the proximal end of the lumens to right atrium). The balloons of the second and third catheters can be inflated to occlude the superior and inferior vena cava, respectively. The distal ends of the lumens of the second and third catheters can be connected to a second external reservoir. To resuscitate a cardiac arrest patient, the resuscitation fluid from the first external reservoir is infused via the lumen of the first catheter into coronary arteries, carrying oxygen and nutrients nourishing the heart tissue. The resuscitation fluid PO₂ from the first catheter shall be at least 100 mmHg. The effluent gathered at right atrium from coronary sinus can then be drained away continuously via the lumens of the second and the third catheter in the right heart into the second external reservoir. The effluent PO₂, which is generally controlled at above 40 mmHg, determines the perfusion flow rate and pressure. The perfusion flow rate can be adjusted by the perfusion pressure. The perfusion flow rate between 1-1500 ml/minute can be used. The perfusion pressure between 1-120 mm Hg is usually needed. It is preferred that the perfusion pressure starts gradually from low pressure to required pressure during perfusion. The pressure can come from the hydrostatic pressure of the first external reservoir, which can be placed above the heart. Optionally, a pump can be used to create the perfusion pressure. The perfusion flow rate generally creates the proper perfusion pressure depending on the size of heart. For an adult human heart, the perfusion flow rate at 100-1,500 ml/minute (i.e., 30-150 ml/minute/100 g cardiac tissue) typically creates 80-100 mm Hg of perfusion pressure. The initial effluent, which contains the mixture of blood, metabolites and resuscitation fluid, can be discarded until the effluent becomes clear. The effluent resuscitation fluid in the second external reservoir can be re-circulated back to the first external reservoir where it is oxygenated with CO₂ and air or O₂, (e.g. the preferred proportion of gas mixture is 5% of CO₂: 95% of air or 95% of O₂.5% CO₂ and 95% atmospheric air or 95% O₂). Accordingly, a kit for resuscitation fluid perfusion can include a resuscitation fluid, at least one catheter (e.g., two or three catheters) having a balloon and a lumen, and at least one pump, (e.g., two pumps), at least one external reservoir (e.g. two external reservoirs), tubes for interconnection and a source of O₂ and CO₂. A low temperature is protective to the heart. The heart beat slows down at a low temperature. It has been reported that heart beat is stopped below 13° C. in a human heart. Therefore, it is preferred that the temperature of the perfused resuscitation fluid is maintained between 13° C.-37° C. for the goal of ROSC. While the resuscitation fluid is continuously perfused, respiratory mechanical ventilation can be initiated as early as possible (e.g., as soon as the perfusion starts). When the ROSC is achieved and stabilized for a short period (for example 1-30 minutes), the heart is generally ready to return to natural physiological mode of blood circulation, which can be established by taking the steps below: the balloons of the second and third catheters in the superior and inferior vena cava are deflated, the balloon of the first catheter in the aorta is deflated, draining of the effluent from the lumens of the second and third catheters is stopped, and the lumen of the first catheter in the aorta is blocked. The central venous pressure is generally maintained at normal level (0-8 mm Hg). Blood can be withdrawn from the second or third catheter in the superior or inferior vena cava if the central venous pressure is high. After the beating heart draws venous blood to enter right atrium and right ventricle, the right ventricle pumps blood to lungs for gas exchange. The blood carrying oxygen leaves the lungs to enter left atrium and left ventricle, the latter pumping the blood into aorta, thereby resuming the blood circulation. After heart beat is stabilized, all catheters can then be removed.

When resuscitation is unable to be started soon after cardiac arrest (e.g., more than 10-30 minutes or even shorter after cardiac arrest if ambient temperature is high or when the ROSC cannot be reached quickly by perfusing the heart alone with a resuscitation fluid), the resuscitation fluid can be perfused to the whole body instead to the heart only. Resuscitation fluid perfusion for the whole body can keep all organs in the whole body viable while waiting for ROSC. This procedure can be achieved by using at least two catheters. For example, a first catheter having a lumen can be introduced from a peripheral artery into the aorta (preferably, the proximal end of the lumen to ascending aorta). The distal end of the lumen can be connected to a first external reservoir where resuscitation fluid is oxygenated continuously with gas mixture of CO₂ and air or O₂. The preferred proportion of gas mixture is 5% of CO₂ and 95% of air or 5% of CO₂ and 95% of O₂. The fluid communication can be established between the first external reservoir and the heart via the first catheter. Optionally, the first external reservoir can be replaced by a bubble or membrane oxygenator and blood reservoir used in cardiopulmonary bypass surgery to oxygenate resuscitation fluid. A second catheter having a lumen can be introduced into venous system (e.g., the femoral vein, vena cava, or right atrium). The resuscitation fluid can then be infused into arterial system to provide oxygen and nutrients for all organs in the body. The blood can be withdrawn and flushed out from the second catheter in venous system. The initial mixture of blood and the resuscitation fluid from venous system can be collected and centrifuged. All blood cells precipitated can be saved. The supernatant (i.e., the diluted plasma) can be treated by a concentration process. For example, a tangential flow filtration apparatus with a membrane of low molecular weight cut off, for example, 500 Daltons. The concentration process eliminates extra water, maintains electrolytes at normal level, and keeps all plasma nutrients with molecular weight above 500 Dalton. After this process, the plasma can be saved for later use. When the effluent from the second catheter becomes clear (i.e., without containing any significant amount of blood), it is directed to a second external reservoir. The effluent PO₂, which is typically controlled at above 40 mm Hg, can be used to determine the perfusion flow rate and the perfusion pressure. The perfusion flow rate generally creates the proper perfusion pressure depending on the size of body weight. For example, the perfusion flow rate between 1-5000 ml/minute can be used. The perfusion pressure between 1-120 mm Hg is usually needed. It is preferred that the perfusion pressure starts gradually from low pressure to the required pressure during perfusion. A pump can be used to create the perfusion pressure. Optionally, the perfusion pressure can derive from the hydrostatic pressure of the first external reservoir when it is placed above the heart. While the resuscitation fluid is perfused for the whole body via the aorta, additional catheter (e.g., a third catheter) can be introduced into the artery of an organ (e.g., heart, lungs, brain, liver, or kidney) to perfuse that organ independently if needed. Clear effluent resuscitation fluid from the second external reservoir can be re-circulated back to the first external reservoir where it is oxygenated continuously with a gas mixture of CO₂ and air or O₂. The kit for resuscitation fluid perfusion of the whole body can include a resuscitation fluid, at least one catheter (e.g., at least two or three catheters), at least two external reservoirs, two pumps, tubes for interconnection, and a source of O₂ and CO₂. Optionally, an external reservoir can be replaced by a bubble or membrane oxygenator and blood reservoir used in cardiopulmonary bypass surgery for oxygenating a resuscitation fluid. Perfusing a cooled resuscitation fluid can be an effective way to lower body temperature which is known to protect tissue. The temperature of the resuscitation fluid can be controlled between 0.1-37° C. It is preferred that the temperature of the resuscitation fluid is maintained between 0.1° C.-13° C. when the ROSC cannot be quickly reached or for long term life support before establishment of ROSC. It has been reported that heart beat is stopped below 13° C. Therefore, it is preferred that the temperature of the resuscitation fluid is maintained between 13° C.-37° C. when it is ready for ROSC. Resuscitation fluid perfusion for the whole body can keep all organs in the body viable while waiting for ROSC. While the resuscitation fluid is continuously perfused, respiratory mechanical ventilation can be initiated as early as possible (e.g., as soon as the perfusion starts). The duration of resuscitation fluid perfusion depends on the establishment of ROCS and availability of the blood. When the ROSC is achieved and stabilized for a period (e.g., 10 minutes to 5 hours), the heart is ready to return to the natural physiological mode of blood circulation, which can be established by taking the following steps: after patient's own plasma that has been treated with concentration process is oxygenated with CO₂ and air or O₂, patient's own blood cells can be carefully added into plasma. Optionally, fresh blood from donors can also be used. The blood can then be perfused into aorta to flush out the resuscitation fluid. When the blood appears in the effluent, draining of the effluent can be stopped. The central venous pressure is generally maintained at normal level (0-8 mm Hg). Blood can be withdrawn from the second catheter in the venous system if central venous pressure is high. The beating heart then draws venous blood to enter the right atrium and right ventricle, which pumps blood to lungs for gas exchange. The blood carrying oxygen leaves the lungs to enter left atrium and left ventricle, the latter pumping the blood to aorta to resume natural physiological mode of blood circulation. After heart beat is stabilized, all catheters can be removed.

When the resuscitation fluid perfusion of the whole body is used to resuscitate intoxication induced cardiac arrest patients (e.g., patients with cardiac arrest induced by alcohol intoxication, drug abuse, medicine overdose, toxins in the blood, contaminant in the blood, sepsis, or venomous snake bite), the perfusion can start even when the heart is still beating. In severe intoxication, cardiac arrest can be inevitable. An advantage of the resuscitation method disclosed herein is that the resuscitation fluid not only provides oxygen and nutrients, but also serves as a vehicle to remove toxin from the body. In such embodiments, a large amount of the resuscitation fluid can be used to flush out the patient's blood as completely as possible, and the resuscitation fluid is not re-circulated until toxin in the effluent is completely removed or until the toxin level falls within the range that is not harmful to the patient. In some embodiments, when a large amount of a toxin is present or a toxin has a wide spread distribution within the body, resuscitation fluid flush may last for a long time (e.g., from five hours to 30 days). If the patient's own blood can be re-used to resume natural physiological mode of blood circulation, one can centrifuge the blood, discard the plasma, wash the isolated blood cells with a saline solution or resuscitation fluid until no toxin can be found, and then adding the blood cells into the resuscitation fluid to be re-used. If the patient's own blood cannot be used, one can use a donor's blood to resume natural physiological mode of blood circulation of a patient.

Resuscitation fluid perfusion for the whole body can also be used to replace blood extracorporeal circulation used during heart open surgery when heart beat is stopped, or during aortic surgery when aorta is cross-clamped.

In one aspect, this disclosure features a method of resuscitating a cardiac arrest patient. The method includes the steps of: A. introducing a first catheter having a lumen and a balloon into the aorta of the patient from a selected peripheral artery, inflating the balloon of the first catheter to block the aorta, and establishing fluid communication between heart coronary arteries and a first external reservoir via the lumen of the first catheter; B. introducing a second catheter have a lumen and a balloon and a third catheter having a lumen and a balloon into the right heart of the patient from selected peripheral veins, inflating the balloons of the second and the third catheter to block superior vena cava and inferior vena cava, and establishing fluid communication between the right heart and a second external reservoir via the lumens of the second and third catheters; C. perfusing the heart with a resuscitation fluid from the first external reservoir via the first catheter, in which the resuscitation fluid includes: Na⁺ 120-155 meq/L, Cl⁻ 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L and water, and the resuscitation fluid has an osmolality of 280-500 mOsm/L, a pH of 7-7.45, PO₂≧100 mm Hg, and PCO₂≦40 mm Hg; D. draining an effluent of the resuscitation fluid into the second external reservoir from the right heart via the second and third catheters; E. circulating the effluent of the resuscitation fluid from the second external reservoir into the first external reservoir where the resuscitation fluid is continuously oxygenated with CO₂ and atmosphere or O₂; and F. deflating the balloons of the first, second and third catheters to resume blood circulation when the patient's heart beat on its own.

In another aspect, this disclosure features a method of resuscitating a cardiac arrest patient that includes the steps of: A. introducing a first catheter having a lumen into the aorta of the patient from a selected peripheral artery to establish fluid communication between the aorta and a first external reservoir via the lumen of the first catheter; B. introducing a second catheter having a lumen into the venous system to establish fluid communication between the venous system and a second external reservoir via the lumen of the second catheter; C. perfusing the patient with a resuscitation fluid from the first external reservoir via the first catheter, in which the resuscitation fluid includes: Na⁺ 120-155 meq/L, 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L and water, and the resuscitation fluid has an osmolality of 280-500 mOsm/L, a pH of 7-7.45, PO₂≧100 mm Hg, and PCO₂≦40 mm Hg; D. draining blood and an effluent of the resuscitation fluid from the venous system into the second external reservoir via the lumen of the second catheter; E. circulating the effluent of the resuscitation fluid from the second external reservoir into the first external reservoir where the resuscitation fluid is continuously oxygenated with O₂ or CO₂ and atmospheric air; and F. infusing blood in the aorta to resume blood circulation when patient's heart beat on its own.

In another aspect, this disclosure features a method of resuscitating a cardiac arrest patient that includes the steps of: A. introducing a first catheter having a lumen and a balloon into the aorta of the patient from a selected peripheral artery, and inflating the balloon to block the aorta; B. introducing a second catheter having a lumen into the right heart of the patient from a selected peripheral vein; C. perfusing the heart with blood via the lumen of the first catheter, and D. withdrawing blood from the right heart via the lumen of the second catheter.

In another aspect, this disclosure features a method of resuscitating a cardiac arrest patient that includes: infusing a resuscitation fluid into the arterial system, and draining an effluent of resuscitation fluid from the venous system. The resuscitation fluid includes: Na⁺ 120-155 meq/L, 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L and water. The resuscitation fluid has an osmolality of 280-500 mOsm/L, a pH of 7-7.45, PO₂≧100 mm Hg, and PCO₂≦40 mm Hg;

In another aspect, this disclosure features a method of resuscitating a cardiac arrest patient that includes: perfusing the heart of a patient with a resuscitation fluid via the aorta, and draining an effluent of the resuscitation fluid from the right heart. The resuscitation fluid includes: Na⁺ 120-155 meq/L, 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L and water, and has an osmolality of 280-500 mOsm/L, a pH of 7-7.45, PO₂≧100 mm Hg, and PCO₂≦40 mm Hg.

In another aspect, this disclosure features a method of providing life support during open heart surgery or aortic surgery of a patient when heart beat is stopped or aorta is cross-clamped. The method includes perfusing the whole body of a patient with a resuscitation fluid and circulating the resuscitation fluid by a cardiopulmonary bypass equipment. The resuscitation fluid comprises: Na⁺ 120-155 meq/L, 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L and water, and has an osmolality of 280-500 mOsm/L, a pH of 7-7.45, PO₂≧100 mm Hg, and PCO₂≦40 mm Hg.

In another aspect, this disclosure features a method of resuscitating a cardiac arrest patient that includes perfusing the whole body of a patient with a resuscitation fluid and circulating the resuscitation fluid by a cardiopulmonary bypass equipment. The resuscitation fluid includes: Na⁺ 120-155 meq/L, 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L and water, and has an osmolality of 280-500 mOsm/L, a pH of 7-7.45, PO₂≧100 mm Hg, and PCO₂≦40 mm Hg.

In still another aspect, this disclosure features a kit of resuscitating a cardiac arrest patient. The kit includes at least a catheter (e.g., two or three catheters) having a lumen, at least a catheter (e.g., two or three catheters) having a balloon and a lumen, at least a reservoir (e.g., two reservoirs), at least a pump (e.g., two pumps), tubes for interconnection, source of O₂ and CO₂, and a resuscitation fluid. The resuscitation fluid includes: Na⁺ 120-155 meq/L, 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L and water.

### BRIEF DESCRIPTION OF DRAWINGS

The following figures depict illustrative embodiments of the invention. These depicted embodiments may not be drawn to scales and are to be understood as illustrative of the invention and not as limiting, the scope of the invention instead being defined by the appended claims. Various advantages of the present invention will become apparent to one skilled in the art by reading the specification and appended claims, and by referencing the following drawings in which:

FIG. 1A is a schematic diagram of a catheter having a balloon and a lumen.

FIG. 1B is a schematic diagram of a catheter having a lumen but without a balloon.

FIG. 2 shows a schematic diagram illustrating an exemplary method of perfusing the heart of a cardiac arrest patient with a resuscitation fluid to resuscitate the patient on the left, and an amplifying schematic diagram depicting the placement of three catheters having a balloon and a lumen to create an isolated perfusing environment around a patient's heart on the right. This method can be used to resuscitate a patient when resuscitation can be initiated soon after cardiac arrest.

FIG. 3 is a schematic diagram illustrating an exemplary method of perfusing the whole body of a cardiac arrest patient with a resuscitation fluid to resuscitate the patient. This method can be used to resuscitate a patient when resuscitation cannot be initiated soon after cardiac arrest, or to resuscitate a patient with intoxication induced cardiac arrest, or to replace extracorporeal blood circulation during heart open surgery when heart beat is stopped or during aortic surgery when aorta is cross-clamped.

### DETAILED DESCRIPTION

### Definitions

For convenience, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e., to at least one) of the grammatical object of the article. By way of example, “an element” means one element or more than one element.

The term “cardiac arrest” is also known as heart arrest, cardiopulmonary arrest, circulatory arrest, ventricular fibrillation, sudden cardiac arrest, sudden death, sudden cardiac death, sudden arrest, asystole, clinical death, or cardioplegic arrest.

The term “cardioplegic arrest” refers to cardiac arrest induced by cardioplegic solution during heart surgery when heart beat need to be stopped. Current cardioplegic solution contains higher amounts of potassium and or magnesium ions.

The term “CPR” refers to cardiopulmonary resuscitation.

The term “CPB” refers to cardiopulmonary bypass. It is a technique that temporarily takes over the function of the heart and lungs during open heart surgery. To repair defects of a heart or an aorta, the surgeon requires a bloodless, motionless operating field to work. To achieve this, the motion of the heart and lungs must be stopped. For this to occur, there needs to be a means for blood to circulate throughout the body, delivering the nutrients and oxygen necessary for life, while the heart and lungs are not functioning. This is made possible through the CPB.

The term “cardiopulmonary bypass equipment” means equipment that currently used for cardiopulmonary bypass. The CPB equipments usually include: (1) a series of tubes made of silicone rubber or PVC for interconnection of CPB circuit, (2) a large cannula to be placed in venous system allowing blood from the body to enter CPB circuit, (3) a large cannula to be placed in arterial system allowing oxygenated blood from CPB circuit to infuse into the body, (3) mechanical pumps such as a roller pump (also known as peristaltic pump) or a centrifugal pump, (4) an oxygenator, and (5) a blood bag or reservoir. A roller pump console usually includes several rotating motor-driven pumps that peristaltically “massage” tubing to propel the blood through the tubing. A centrifugal pump produces blood flow by centrifugal force. An oxygenator performs the same jobs as the lungs, i.e. providing oxygen to the blood and removing carbon dioxide from the blood. There are typically two types of oxygenator, i.e. bubble oxygenator and membrane oxygenator.

The term “ROSC” refers to the Return of Spontaneous Circulation, meaning the heart starts to beat on its own again after cardiac arrest.

The term “PO₂” refers to partial oxygen pressure. The term “PCO₂” refers to partial carbon dioxide pressure.

The term “resuscitation” means a treatment effort to bring the patient back to life.

The term “perfuse” means delivery of a fluid into patient or its organ via vascular system such that the fluid reaches the entire body or organ of the patient.

The term “infuse” means delivery of a fluid into a patient's vascular system.

The term “right heart” includes right atrium, right ventricle, superior vena cava and inferior vena cava.

The term “peripheral artery” refers to any arteries outside the heart and the aorta.

The term “peripheral vein” refers to any veins outside the heart, superior vena cava and inferior vena cava.

The term “central venous pressure” refers to the pressure of blood in the thoracic vena cava, near the right atrium of the heart.

The term “physiological saline based fluid” refers to the fluid that contains 0.9 wt % sodium chloride and optionally other nutrients. It includes Krebs-Henseleit solution, Tyrode solution, Locke solution, Lactated Ringer's solution, Ringer's solution, serum, even cell culture medium.

The term “serum” is also known as blood serum. It is collected after blood coagulation. It contains all components of blood except blood cells and clotting factors.

The term “cell culture medium” refers to a liquid or gel designed to support growth of cells derived from animals. It usually contains inorganic salts, amino acids, vitamins, glucose, insulin, plasma protein (or serum).

The terms “comprise” and “comprising” are used in the inclusive, open sense, meaning that additional elements may be included.

The term “including” is used to mean “including but not limited to”. “Including” and “including but not limited to” are used interchangeably.

A “patient” to be treated by the subject method refers to either a human or non-human animal.

The term “treating” is art-recognized and refers to curing as well as ameliorating at least one symptom of any condition or disease.

All organs in the body depend on oxygen and nutrients for their viability. Like the blood, many physiological saline based fluids, such as Krebs-Henseleit solution, Tyrode solution, Ringer's solution, lactated Ringer's solution, serum and cell culture medium, and their variants etc. can also carry a certain amount of oxygen. The inventor has unexpectedly found that, although these physiological saline based fluids carry a low level of oxygen, they can still be used as a resuscitation fluid to replace blood to keep organ's viability during cardiac arrest. The O₂ can dissolve in the resuscitation fluid as it expose to the air or O₂. The PO₂ of the resuscitation fluid can reach 154-158 mmHg if it is exposed to normal atmosphere air at sea level. It is calculated that this can provide O₂ of 0.45 ml/100 ml of the resuscitation fluid. The PO₂ of the resuscitation fluid can reach 500-600 mmHg if it is exposed to the 95% O₂. It is calculated that this can provide O₂ of 1.5 ml/100 ml of resuscitation fluid.

The inventor has found the composition of optimal resuscitation fluid (which is analogous to serum) as follow: Na⁺ 120-155 meq/L, 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L and water. The composition typically has an osmolality between 280-500 mOsm/L, a pH between 7-7.45, PO₂ above 100 mm Hg, and PCO₂ below 40 mm Hg. An example of the resuscitation fluid includes Na 130 meq/L, Cl⁻ 140 meq/L, K⁺ 3.5 meq/L, Ca 2.5 meq/L, Mg 2 meq/L, HCO₃⁻ 25 meq/L, Glucose 100 mg/dl, albumin 0.1 gram/dl, insulin 10 IU/L, heparin 10 U/L and water.

Other examples of the resuscitation fluid include cell culture media include the following ingredients: Na⁺ 120-155 meq/L, Cl⁻ 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L, L-Arginine.HCl 0-0.2 g/L, L-Cystine.2HCl 0-0.2 g/L, L-Glutamine 0-0.2 g/L, Glycine 0-0.2 g/L, L-Histidine.HCl.H₂O 0-0.2 g/L, L-Isoleucine 0-0.2 g/L, L-Leucine 0-0.2 g/L, L-Lysine.HCl 0-0.2 g/L, L-Methionine 0-0.2 g/L, L-Phenylalanine 0-0.2 g/L, L-Serine 0-0.2 g/L, L-Threonine 0-0.2 g/L, L-Tryptophan 0-0.2 g/L, L-Tyrosine.2Na.2H₂O 0-0.2 g/L, L-Valine 0-0.2 g/L, Choline Chloride 0-0.01 g/L, Folic Acid 0-0.01 g/L, myo-Inositol 0-0.01 g/L, Nicotinamide 0-0.01 g/L, D-Pantothenic Acid 0-0.01 g/L, Pyridoxine.HCl 0-0.01 g/L, Riboflavin 0-0.001 g/L, Thiamine.HCl 0-0.01 g/L, Vitamin B-12 0-0.001 g/L. The pH of the resuscitation fluids can be about 7-7.45 when it is continuously oxygenated with gas mixture of CO₂ and air or O₂. The preferred proportion of gas mixture is 5% of CO₂ and 95% of air or 5% of CO₂ and 95% of O₂. The cell culture medium is most preferred for long term perfusion, e.g. perfusion for several days to several months. An example is the DMEM cell culture medium which includes the following: NaCl 6.4 g/L CaCl₂ 0.2 g/L, MgSO₄ 0.0977 g/L, KCl 0.4/L, NaHCO₃ 1.5 g/L, NaH₂PO₄.H₂O 0.125 g/L, L-Arginine.HCl 0.084 g/L, L-Cystine.2HCl 0.0626 g/L, L-Glutamine 0.584 g/L, Glycine 0.03 g/L, L-Histidine.HCl.H₂O 0.042 g/L, L-Isoleucine 0.105 g/L, L-Leucine 0.105 g/L, L-Lysine.HCl 0.146 g/L, L-Methionine 0.03 g/L, L-Phenylalanine 0.066 g/L, L-Serine 0.042 g/L, L-Threonine 0.095 g/L, L-Tryptophan 0.016 g/L, L-Tyrosine.2Na.2H₂O 0.10379 g/L, L-Valine 0.094 g/L, Choline Chloride 0.004 g/L, Folic Acid 0.004 g/L, myo-Inositol 0.0072 g/L, Nicotinamide 0.004 g/L, D-Pantothenic Acid 0.004 g/L, Pyridoxine.HCl 0.004 g/L, Riboflavin 0.0004 g/L, Thiamine.HCl 0.004 g/L, Vitamin B-12 0.00068 g/L, D-Glucose 1 g/L, Insulin 4 mg/L, and Albumin 2.5 g/L).

The pH of the resuscitation fluid can be about 7-7.45 when it is continuously oxygenated with gas mixture of CO₂ and air or O₂. The preferred proportion of gas mixture is 5% of CO₂ and 95% of air or 5% of CO₂ and 95% of O₂.

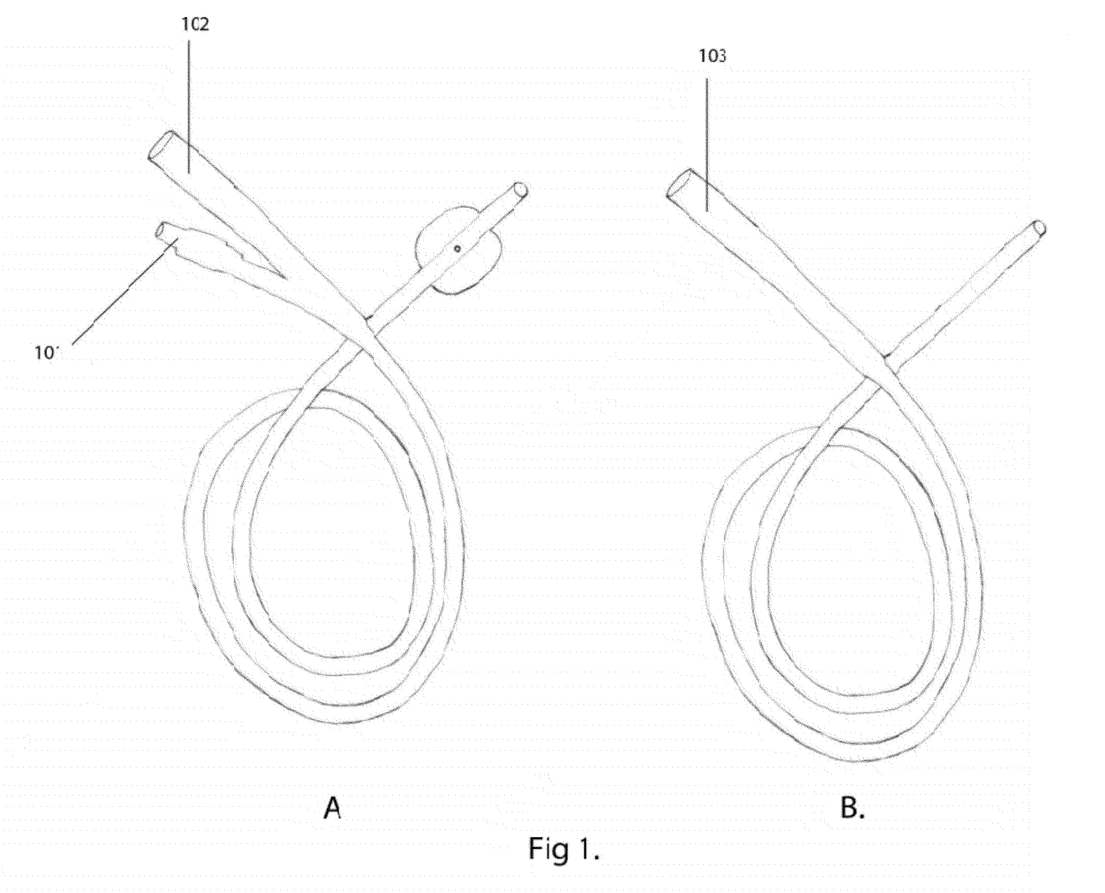

FIG. 1 shows a schematic diagram of catheters used to infuse or drain the resuscitation fluid in this disclosure. FIG. 1A is a catheter having a balloon and a lumen. As shown in FIG. 1A, 101 represents a port that can be used to inflate the balloon, and 102 represents a port that can be used to infuse or drain the resuscitation fluid. The inflated balloon has a size such that it is generally able to occlude the aorta, superior vena cava, and inferior vena cava. Typically, in a human adult, the diameter of ascending aorta is about 27-36 mm, the diameter of an inferior vena cava is about 15-25 mm, and the diameter of a superior vena cava is about 15-30 mm. The catheter size can be small enough to be inserted into a peripheral artery and a vein. For example, 1 French gauge (0.33 mm diameter) to 8 French gauge (2.7 mm diameter) catheters can be selected. The catheter having a balloon and a lumen described in FIG. 1A is available from numerous commercial sources. Examples include Berenstein lumen occlusion balloon catheters, Equalizer occlusion balloon catheters (Boston Scientific), Foley balloon catheters (Sterimed Medical Devices Pvt. Ltd), and Forgarty occlusion catheters (Edwards Lifesciences). FIG. 1B is a catheter having a lumen (without a balloon). As shown in FIG. 1B, 103 represents a port that can be used to infuse or drain the resuscitation fluid. The catheter size can be small enough to be inserted into a peripheral artery and a vein. For example, 1 French gauge (0.33 mm diameter) to 8 French gauge (2.7 mm diameter) catheters can be selected. The catheter described in FIG. 1B is available from numerous commercial sources.

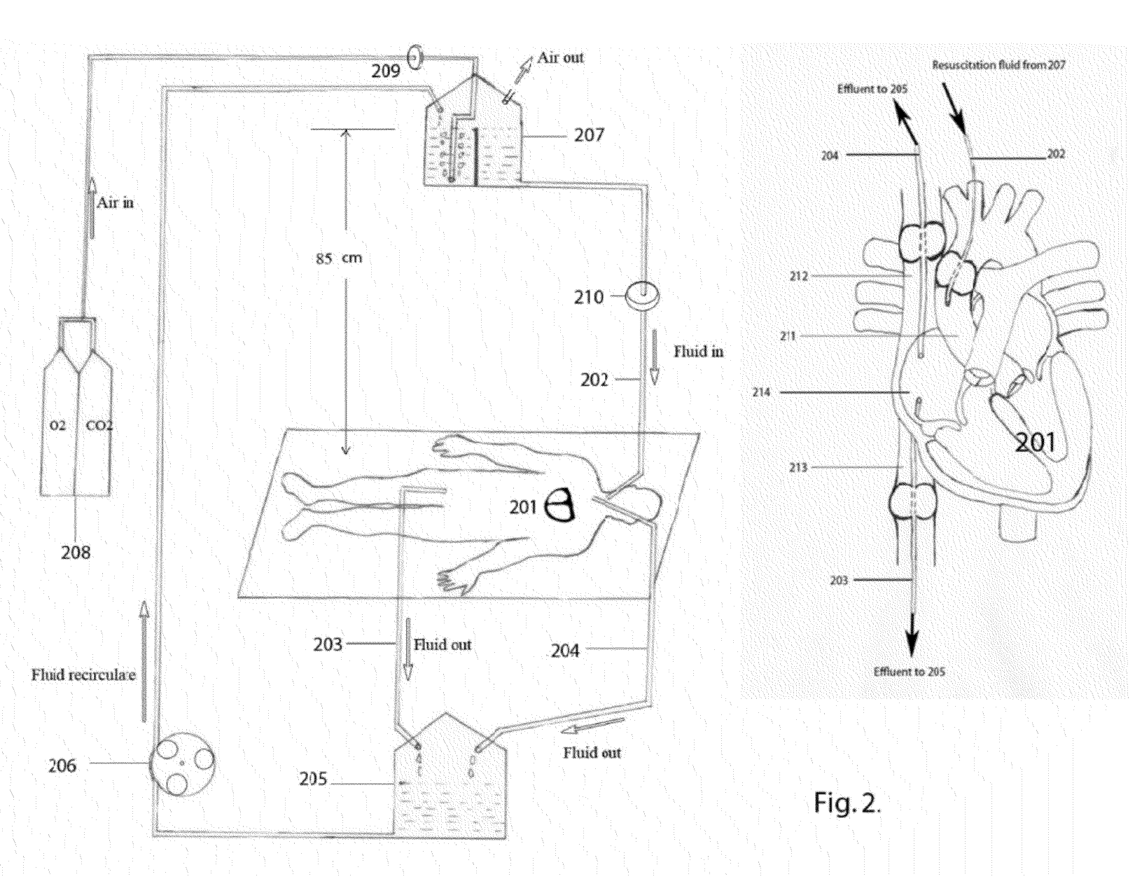

When resuscitation is able to be started soon after cardiac arrest, a resuscitation fluid can be perfused exclusively to the heart, for example, within 10-30 minutes or even hours if ambient temperature is low. Under this circumstance, quick establishment of ROSC shall be the main goal. This can be accomplished by creating an isolated environment solely to perfuse the heart with a resuscitation fluid via three catheters having a balloon and a lumen described in FIG. 1A. Examples of this method are illustrated in FIG. 2.

FIG. 2 shows a schematic diagram illustrating an exemplary method of perfusing the heart of a cardiac arrest patient with a resuscitation fluid to resuscitate the patient on the left, and an amplifying schematic diagram depicting the placement of three catheters having a balloon and a lumen to create an isolated perfusing environment around a patient's heart on the right. In FIG. 2, 201 represents the heart of a patient, 202 represents a first catheter having a balloon and a lumen, 204 represents a second catheter having a balloon and a lumen, 203 represents a third catheter having a balloon and a lumen, 205 represents a second external reservoir, 206 represents a pump, 207 represents a first external reservoir, 208 represents a gas mixture tank, 209 represents a filter, 210 represents a filter and air bubble trap, 211 represents ascending aorta, 212 represents superior vena cava, 213 represents inferior vena cava, and 214 represents right atrium.

As shown in FIG. 2, to resuscitate a patient with arrested heart 201, a first catheter 202 having a balloon and a lumen is introduced from a peripheral artery into the ascending aorta 211 to provide an inflow of the resuscitation fluid. A second catheter 204 having a balloon and a lumen is introduced from right jugular vein or subclavian vein into right heart via superior vena cava 212. A third catheter 203 having a balloon and a lumen is introduced from femoral vein into right heart via inferior vena cava 213. The second 204 and third 203 catheters are to provide outflow of the resuscitation fluid. The balloons of these three catheters can be inflated to occlude the ascending aorta 211, superior vena cava 212 and inferior vena cava 213. With these three catheters having a balloon and a lumen in place, an isolated perfusing environment of the heart can be created.

The lumen of the distal end of the first catheter 202 is connected to a first external reservoir 207 and the lumen of the proximal end of the first catheter 202 is open to the ascending aorta between the inflated balloon and the heart. The proximal ends of the second 204 and third 203 catheters are open to right atrium 214 and the distal ends of the second 204 and third 203 catheters are connected to a second external reservoir 205.

The resuscitation fluid from the first external reservoir 207 can be perfused in the retrograde direction down the ascending aorta via the first catheter 202 into coronary arteries, carrying oxygen and nutrients to nourish the heart tissue. A 0.22 μm filter 210 can be optionally installed to sterilize the resuscitation fluid before it reaches the heart tissue. The filter 210 can also be served as a bubble trap to eliminate possible air bubble in the resuscitation fluid.

The effluent of the resuscitation fluid gathered at right atrium 214 from coronary sinus is then drained through the second and third catheters 204 and 203 into the second external reservoir 205. The PO₂ of the effluent, which is typically controlled at above 40 mm Hg, can be used to determine the perfusion flow rate and pressure. The perfusion flow rate is adjusted to create the right amount of the perfusion pressure depending on the size of the heart. The perfusion pressure between 1-120 mm Hg is usually needed inside the aorta. It is preferred that the perfusion pressure starts gradually from a low pressure to the required pressure within a period of, for example, 10 minutes to 3 hours. The perfusion pressure comes from the constant hydrostatic pressure of the first external reservoir 207, which can be placed, for example, 85 centimeters above heart level. Optionally, a pump, such as a peristaltic pump or a centrifugal pump, can be used to create the perfusion pressure. For an adult human heart, the perfusion flow rate at 100-1,500 ml/minute creates a perfusion pressure of 80-100 mm Hg or 30-150 ml/minute/100 g cardiac tissue.

Typically, the initial effluent, which contains the mixture of blood, metabolites and the resuscitation fluid, is discarded until it becomes clear. The effluent of the resuscitation fluid is then delivered to the second external reservoir 205, and then is transferred with a pump 206 to the first external reservoir 207 where it is oxygenated by mixing with a gas mixture 208 containing O₂ and CO₂ (e.g., 95% O₂ and 5% CO₂ or 95% atmospheric air and 5% CO₂). A 0.22 μm filter 209 can be optionally used to sterilize the gas mixture. The first external reservoir 207 can have two compartments. The first compartment receiving the resuscitation fluid and gas mixture 208 contains lots of air bubbles. The first compartment generally serves as an oxygenator to mix gas mixture 208 with the resuscitation fluid. The resuscitation fluid contained in the second compartment is overflowed from the first compartment and contains no bubbles. The second compartment generally serves as a resuscitation fluid storage. Optionally, a blood reservoir and an oxygenator (for example, a membrane oxygenator or bubble oxygenator) used for cardiopulmonary bypass during cardiac surgery can be used to function as the first external reservoir 207.

The temperature of the resuscitation fluid can be controlled between 0.1-37° C. Low temperature is protective to the heart as heart beat slows down as the temperature decreases. It has been reported that heart beat is stopped below 13° C. Therefore, it is preferred that the temperature of the resuscitation fluid is maintained between 13° C.-37° C. to achieve ROSC.

In general, while the heart is perfused with the resuscitation fluid, respiratory mechanical ventilation is initiated as early as possible.

When the ROSC is achieved and stabilized for a short period (e.g., 1-30 minutes), the heart is ready to return to natural physiological mode of blood circulation. The balloons from the second catheter 204, the third catheter 203, and the first catheter 202 can then be deflated, the draining of the effluent from the second 204 and the third 203 catheters is stopped, and then the perfusion of the heart with the resuscitation fluid from the first catheter 202 can be stopped. The central venous pressure can be maintained at a level of 0-8 mm Hg. The blood can be withdrawn from the second 204 and or third 203 catheters in the superior and or inferior vena cava if the central venous pressure is high. The beating heart draws venous blood to enter right atrium and right ventricle, then right ventricle pumps blood to lungs for gas exchange. The blood carrying oxygen leaves the lungs to enter left atrium and left ventricle. The left ventricle then pumps the blood to aorta and therefore resumes the blood circulation. After heart beat is stabilized, all catheters can then be removed.

In some embodiments, when it is relatively easy to resuscitate a cardiac arrest patient (e.g., when the patient is resuscitated immediately after cardiac arrest), the resuscitation of the heart can be simple. The second catheter 204 and the third catheter 203 may be omitted, and the second external reservoir 205 and the pump 206 may also be omitted. Instead, one catheter, for example, a catheter without a balloon described in FIG. 1B, can be introduced into the right heart, e.g. right atrium 214, from a peripheral vein. The ROSC can be soon achieved by perfusing the heart with the resuscitation fluid, or optionally, donor blood if available, via the first catheter 202 in the aorta and withdrawing a mixture of blood and resuscitation fluid via the catheter without a balloon in right heart. The mixture of the blood and resuscitation fluid can be discarded.

In some embodiments, three catheters having a balloon and a lumen can connect to a cardiopulmonary bypass equipment to perfuse the heart with the resuscitation fluid.

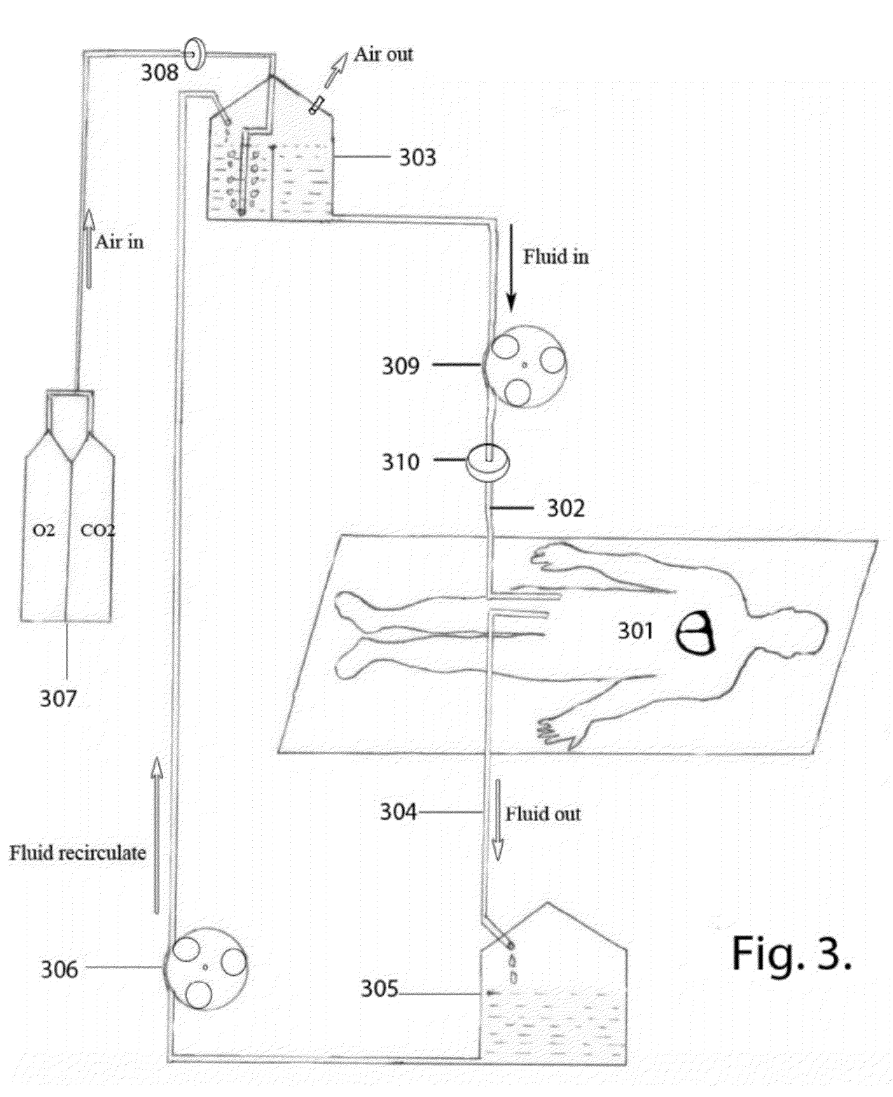

Resuscitation fluid perfusion of the whole body is suitable when resuscitation is unable to be started soon after cardiac arrest, for example, more than 30 minutes or even shorter if ambient temperature is high or when the ROSC cannot be quickly resumed with resuscitation fluid perfusion of heart. Resuscitation fluid perfusion of the whole body can keep all organs in the whole body viable and gradually resume functions while waiting for ROSC. Perfusion of the whole body with a resuscitation fluid can be accomplished by two catheters having a lumen described in FIG. 1B. Exemplary method of perfusing the whole body with a resuscitation fluid is illustrated in FIG. 3.

In FIG. 3, 301 represents the heart of a patient, 302 represents a first catheter having a lumen, 304 represents a second catheter having a lumen, 303 represents a first external reservoir, 305 represents a second external reservoir, 306 represents a pump, 307 represents a gas mixture tank, 308 represents a filter, 309 represents a pump, and 310 represents a filter and air bubble trap.

To resuscitate a patient with arrested heart 301, the first catheter 302 described in FIG. 1B is introduced from peripheral artery into the aorta, for example, from femoral artery to ascending aorta, to provide an inflow of the resuscitation fluid. The proximal end of the lumen of the first catheter 302 is open to the aorta, and the distal end of the lumen is connected to the first external reservoir 303. A second catheter 304 described in FIG. 1B is introduced into venous system, for example, femoral vein or right atrium.

The resuscitation fluid can be perfused from the first external reservoir 303 into aorta with a peristaltic pump 309, carrying oxygen and nutrients to all organs in the body. A 0.22 μm filter 310 can be optionally installed to sterilize the resuscitation fluid before it reaches the patient. A bubble trap 310 can be optionally installed to eliminate possible air bubble in the resuscitation fluid. The blood can be drained and flushed out from the second catheter 304.

The initial mixture of blood and the resuscitation fluid from the second catheter 304 can be collected and centrifuged so that all blood cells are precipitated and saved. The supernatant (which is the plasma) can be concentrated by a filtration apparatus with a membrane of low molecular weight cut off, such as 500 Daltons. For example, tangential flow filtration apparatus can be used to concentrate the plasma to normal volume. The concentration process eliminates extra water, maintains electrolytes at a normal level, and keeps all plasma nutrients with molecular weight above 500 Dalton. After this process, the plasma can be saved for later use.

As the effluent from the second catheter 304 gradually becomes clear, it goes into the second external reservoir 305. The PO₂ of the effluent, which is typically controlled at above 40 mm Hg, can be used to determine the perfusion flow rate and pressure. The perfusion flow rate is typically adjusted to create the right amount of perfusion pressure depending on the size of body weight. The perfusion pressure between 1-120 mm Hg is usually needed inside the aorta. It is preferred that the perfusion pressure starts gradually from a low pressure to a desired pressure within a period of, e.g., 10 minutes to 3 hours. The pump 309 can be used to create the perfusion pressure. Optionally, the perfusion pressure can derive from the hydrostatic pressure of the first external reservoir 303, which is typically above the heart level (e.g., 85 cm above the heart level). While the resuscitation fluid can be used to perfuse the whole body via the aorta, an additional catheter (not shown in FIG. 3) can be introduced into the artery of an organ (e.g., the heart, lungs, brain, liver, and kidney) and perfuse independently if needed. For an adult human resuscitation, the perfusion flow rate at 1-5,000 ml/minute can be used. A clear effluent of the resuscitation fluid from the second external reservoir 305 can be transferred with the pump 306 to the first external reservoir 303 where the resuscitation fluid is oxygenated with a gas mixture containing O₂ and CO₂ (e.g., 95% O₂ and 5% CO₂ or 95% atmospheric air and 5% CO₂) from the gas mixture tank 307. A 0.22 μm filter 308 can optionally be used to sterilize the gas mixture. The first external reservoir 303 can be the same as reservoir 207 described in FIG. 2.

The temperature of the resuscitation fluid can be controlled between 0.1-37° C. Low temperature can be protective to all organs. Patient's body temperature can be lowered effectively when the body of a patient is being perfused with the resuscitation fluid. It is preferred that the body temperature can be maintained between 0.1° C.-13° C. when ROSC cannot be quickly resumed or when long term life support is needed before establishment of ROSC. It has been reported that heart beat is stopped below 13° C. in human being. Therefore, it is preferred that the temperature of the resuscitation fluid is maintained between 13° C.-37° C. when the heart is ready for ROSC.

While the resuscitation fluid is perfused, respiratory mechanical ventilation can be initiated as early as possible.

The duration of the resuscitation fluid perfusion depends on the ROSC and availability of the blood. When the ROSC is achieved and stabilized for a period (e.g., from 10 minutes to several hours), the heart is ready to return to natural physiological mode of blood circulation. Typically, after a patient's own plasma that has been treated with filtration process is gassed with O₂ and CO₂ (e.g., 95% O₂ and 5% CO₂ or 95% atmospheric air and 5% CO₂), the patient's own blood cells can be carefully added into plasma. Optionally, donor blood can also be used. The blood is then perfused into aorta via the first catheter 302 to flush out the resuscitation fluid in the patient. When the blood appears in the effluent in the second catheter 304, draining of the effluent can be stopped. The central venous pressure can be maintained at a normal level (0-8 mm Hg). The blood can be withdrawn from the second catheter 304 in the venous system if the central venous pressure is high. The beating heart draws venous blood, which enters right atrium and right ventricle. The right ventricle then pumps blood to lungs for gas exchange. The blood carrying oxygen leaves the lungs to enter left atrium and left ventricle. The left ventricle pumps the blood to aorta and therefore resume physiological mode of blood circulation. After heart beat is stabilized, all catheters can be removed.

When resuscitation fluid perfusion of the whole body is used to resuscitate various intoxication induced cardiac arrest patients, such as alcohol intoxication, drug abuse, medicine overdose, toxins in the blood, contaminant in the blood, sepsis, and venomous snake bite, it can start even the heart is still beating. The advantage of this approach is that the resuscitation fluid not only provides oxygen and nutrients, but also serves as a vehicle to remove toxin from the body. Therefore, more resuscitation fluid has to be used to flush out the patient's blood as completely as possible, and the resuscitation fluid is not re-circulated until toxin in the effluent is completely removed or toxin level within the range that is not harmful to the patient. Since some toxins may have large amount and wide spread distribution, resuscitation fluid flush may last for long time (e.g., several hours to several months). The cell culture medium is most preferred for long term perfusion. If the patient's own blood can be re-used to resume physiological mode of blood circulation, one can centrifuge the blood, discard the plasma, and wash the blood with saline or the resuscitation fluid until no toxin can be found. The blood cells can then be added into the resuscitation fluid to be re-used. If the patient's own blood cannot be used, a donor's blood can be used to resume physiological mode of blood circulation.

Perfusing the whole body with a resuscitation fluid can also be used as a life support for open heart surgery when heart beat is stopped, and for aortic surgery when aorta is cross-clamped. The CPB equipment can also be used, for example, bubble oxygenator (Kewei Rising Medical Corporation Limited), Apex hollow fiber membrane blood oxygenator with hardshell venous reservoir, can be selected. Perfusion of the whole body with a resuscitation fluid is advantageous since it is much simpler and without concern of blood cell damage compared with blood perfusion.

### EXEMPLIFICATION

The disclosure now being generally described, it will be more readily understood by reference to the following examples which are included merely for purposes of illustration of certain aspects and embodiments of the present invention, and are not intended to limit the invention in any way.

### Example 1

### Resuscitation Fluid Perfusion of Heart to Treat Cardiac Arrest in a Pig Model

Preparation: Four male pigs (the first pig weighing 45 kg, the second pig weighing 44 kg, the third pig weighing 46 kg, and the fourth pig weighing 47 kg) were fasted overnight but provided water ad libitum before the resuscitation procedure. Cardiac arrest was induced in each pig as follow: Ketamine 20 mg/kg im was given for anesthetic induction. The trachea was incubated and connected to a ventilator (tidal volume 15 ml/kg, rate 15/min, O₂ concentration 30%). 2% isoflurane was given for anesthetic maintenance. The resuscitation process was conducted in room temperature 22-23° C. The left femoral vein and artery was catheterized via an inguinal cutdown. The venous catheter was used for administration of fluids and pharmacological agents. The arterial catheter was used for monitoring blood pressure. Cardiac rhythm was monitored with a standard lead II electrocardiogram (EKG). 5000 U heparin was administered. The pigs were paralyzed with 8 mg/kg pancuronium. Asphyxial cardiac arrest was induced by clamping of the endotracheal tube and stopping of the ventilator. Cardiac arrest was determined by isoelectric EKG.

Resuscitation with conventional CPR: At 30 minutes after cardiac arrest, the first pig was resuscitated with conventional CPR as follow: Mechanical ventilation was resumed (tidal volume 15 ml/kg, rate 15/min, O₂ concentration 100%), and manual closed-chest CPR was initiated. Chest compressions were performed at a rate of 100 compressions/minute for 60 minutes. At 3 minutes after chest compressions, epinephrine was administered iv at 0.02 mg/kg. At 5 minutes after chest compressions, defibrillation was initiated with 100 j. At 10 minutes after chest compressions, epinephrine was administered iv at 0.04 mg/kg again. At 15 minutes after chest compressions, the defibrillation was given again with 200 j. As the ROSC was not achieved within 60 minutes of conventional CPR, the resuscitation attempt was given up.

Resuscitation with Resuscitation fluid perfusion of heart by three balloon catheters and two external reservoirs: At 30 minutes after cardiac arrest, the second pig was resuscitated by perfusing its heart with a resuscitation fluid using three catheters having a balloon and a lument as follow: The first catheter (7 F EQUALIZER occlusion balloon catheter, Boston Scientific, catalog #17-109), which was pre-filled with a resuscitation fluid, was introduced from external carotid artery into the ascending aorta near the heart. The balloon was then inflated with about 19 ml of saline to occlude the ascending aorta. The balloon diameter was about 33 mm. The distal end of the lumen was connected to a first external reservoir, and the proximal end of the lumen was open to the aorta between the inflated balloon and the heart. The second catheter having a balloon and a lumen (7 F EQUALIZER occlusion balloon catheter, Boston Scientific, catalog #17-105) was introduced from right jugular veins into right atrium via superior vena cava. The balloon was inflated with about 4.8 ml of saline to occlude the superior vena cava. The balloon diameter is about 20 mm. The proximal end of the lumen was open to right atrium, and the distal end of the lumen was connected to a second external reservoir. The third catheter having a balloon and a lumen (7 F EQUALIZER occlusion balloon catheter, Boston Scientific, catalog #17-105) was introduced from right femoral vein into right atrium via inferior vena cava. The balloon was inflated with about 4.8 ml of saline to occlude the inferior vena cava. The balloon diameter was about 20 mm. The proximal end of the lumen was open to right atrium, and the distal end of the lumen was connected to the second external reservoir. About 5 liters of a resuscitation fluid containing: NaCl 118.5 mM, NaHCO₃ 25.0 mM, KCl 3 mM, MgSO₄ 2.5 mM, CaCl₂ 2.5 mM, Glucose 100 mg/dl, albumin 0.1 gram/dl, insulin 10 IU/L, heparin 10 U/L, and water was stored in a reservoir where it was oxygenated with 95% O₂ and 5% CO₂. The temperature of the resuscitation fluid is maintained at 25-30° C. The reservoir was hanged above the heart and connected with the first catheter having a balloon and a lumen inside the aorta. The resuscitation fluid from the first external reservoir was perfused into ascending aorta and coronary arteries of the heart, carrying oxygen and nutrients to nourish the heart tissue. The perfusion was initiated at a pressure resulted by placing the reservoir at 10 cm above the heart for 10 minutes. The perfusing pressure was then gradually increased by elevating height of the first external reservoir. Finally, the height of the first reservoir was raised to 85 cm above the heart level over a period of 10 minutes. The effluent gathered at right atrium from coronary sinus was drained from the second and third catheter having a balloon and a lumen. The initial 200 ml of effluent containing a mixture of blood and the resuscitation fluid was discarded. The clear effluent was then drained continuously down into the second external reservoir. The effluent resuscitation fluid was circulated back to the first external reservoir where it was oxygenated. The first cardiac electric activity (QRS wave) was noticed at about 15 minutes after perfusion of the heart with the resuscitation fluid. Then respiratory mechanical ventilation was resumed (tidal volume 15 ml/kg, rate 15/min, O₂ concentration 100%) at 15 minutes after perfusion of the resuscitation fluid. The heart rhythm reached about 40/minutes at 40 minutes after perfusion of the resuscitation fluid. After the balloons in the superior, inferior vena cava and in the aorta were simultaneously deflated, the draining of effluent in the right atrium and perfusing of the resuscitation fluid in the aorta were stopped. The heart rhythm was about 60 beats/minutes at 60 minutes after perfusion of resuscitation fluid. The O₂ concentration in respiratory mechanical ventilation was changed from 100% to 30% for 10 minutes, then changed to room air. At 90 minutes after perfusion of resuscitation fluid, the heart rhythm was about 63 beats/minutes and the blood pressure was 100/60 mm Hg. All catheters were then removed. Three hours after perfusion of resuscitation fluid, the heart rhythm was about 66 beats/minutes and the blood pressure was 110/70 mm Hg. The ROSC was considered successful.

Resuscitation with Resuscitation fluid perfusion of heart by two balloon catheters and one external reservoir: At 30 minutes after cardiac arrest, the third pig was resuscitated by perfusing its heart with a resuscitation fluid using two catheters as follow: The first catheter having a balloon and a lumen (7 F EQUALIZER occlusion balloon catheter, Boston Scientific, catalog #17-109), which was pre-filled with a resuscitation fluid, was introduced from external carotid artery into the ascending aorta near the heart. The balloon was then inflated with about 19 ml of saline to occlude the ascending aorta. The balloon diameter is about 33 mm. The distal end of the lumen was connected to a first external reservoir, and the proximal end of the lumen was open to the aorta between the inflated balloon and the heart. The second catheter without balloon (7 F) was introduced from right jugular veins into right atrium via superior vena cava. About 10 liters of a resuscitation fluid containing NaCl 118.5 mM, NaHCO₃ 25.0 mM, KCl 3 mM, MgSO₄ 2.5 mM, CaCl₂ 2.5 mM, Glucose 100 mg/dl, albumin 0.1 gram/dl, insulin 10 IU/L, heparin 10 U/L, and water was stored in the first external reservoir and oxygenated with 95% O₂ and 5% CO₂. The temperature of the resuscitation fluid was maintained at 25-30° C. The first external reservoir was hanged above the heart and connected with the first catheter having a balloon and a lumen. The resuscitation fluid from the first external reservoir was perfused into ascending aorta and coronary arteries of the heart, carrying oxygen and nutrients to nourish the heart tissue. The perfusion was initiated at a pressure resulted by placing the first external reservoir at 10 cm above the heart for 10 minutes. The perfusing pressure was then gradually increased by elevating the height of the first external reservoir. Finally, the height of the first external reservoir was raised to 85 cm above the heart over a period of 10 minutes. The effluent gathered at right atrium from coronary sinus was withdrawn from the second catheter in the right atrium. The effluent containing a mixture of blood and the resuscitation fluid was continuously withdrawn and discarded. The first cardiac electric activity (QRS wave) was noticed at about 14 minutes after perfusion of the resuscitation fluid. The respiratory mechanical ventilation was resumed (tidal volume 15 ml/kg, rate 15/min, O₂ concentration 100%) at 14 minutes after perfusion of the resuscitation fluid. The heart rhythm reached about 40/minutes at 38 minutes after perfusion of the resuscitation fluid. After the balloon of the first catheter was deflated, the draining of the effluent from the second catheter in the right atrium and the perfusion of the resuscitation fluid in the aorta were stopped. The heart rhythm was about 60 beats/minutes at 60 minutes after perfusion of the resuscitation fluid. O₂ concentration in respiratory mechanical ventilation was then changed from 100% to 30% for 10 minutes, and subsequently changed to atmospheric air. At 90 minutes after perfusion of the resuscitation fluid, the heart rhythm was about 70 beats/minutes and the blood pressure was 107/62 mm Hg. All catheters were then removed. Three hours after perfusion of the resuscitation fluid, the heart rhythm was about 64 beats/minutes and the blood pressure was 101/62 mm Hg. The ROSC was considered successful.

Resuscitation with Resuscitation fluid perfusion of heart with three balloon catheters and CPB equipment: At 30 minutes after cardiac arrest, the fourth pig was resuscitated by perfusing its heart with a resuscitation fluid using three catheters having a balloon and a lumen and CPB equipment as follow: The first catheter having a balloon and a lumen (7 F EQUALIZER occlusion balloon catheter, Boston Scientific, catalog #17-109), which was pre-filled with a resuscitation fluid, was introduced from external carotid artery into the ascending aorta near the heart. The balloon was then inflated with about 19 ml of saline to occlude the ascending aorta. The balloon diameter was about 33 mm. The distal end of the lumen was connected to an APEX hollow fiber membrane oxygenator with hardshell reservoir (catalog #050303, Sorin Group USA Inc.), and the proximal end of the lumen was open to the aorta between the inflated balloon and the heart. The second catheter having a balloon and a lumen (7 F EQUALIZER occlusion balloon catheter, Boston Scientific, catalog #17-105) was introduced from right jugular veins into right atrium via superior vena cava. The balloon was inflated with about 4.8 ml of saline to occlude the superior vena cava. The balloon diameter is about 20 mm. The proximal end of the lumen was open to right atrium, and the distal end of the lumen was connected to a second external reservoir used for CPB. The third catheter having a balloon and a lumen (7 F EQUALIZER occlusion balloon catheter, Boston Scientific, catalog #17-105) was introduced from right femoral vein into right atrium via inferior vena cava. The balloon was inflated with about 4.8 ml of saline to occlude the inferior vena cava. The balloon diameter is about 20 mm. The proximal end of the lumen was open to right atrium, and the distal end of the lumen was connected to the second external reservoir used for CPB. About 4 liters of a resuscitation fluid containing NaCl 118.5 mM, NaHCO₃ 25.0 mM, KCl 3 mM, MgSO₄ 2.5 mM, CaCl₂ 2.5 mM, Glucose 100 mg/dl, albumin 0.1 gram/dl, insulin 10 IU/L, heparin 10 U/L, and water was oxygenated with 95% O₂ and 5% CO₂ in a APEX hollow fiber membrane oxygenator with hardshell reservoir. The temperature of the resuscitation fluid was maintained at 25-30° C. The APEX hollow fiber membrane oxygenator with hardshell reservoir was hanged above the heart. The resuscitation fluid from APEX hollow fiber membrane oxygenator with hardshell reservoir was perfused into ascending aorta and coronary arteries of the heart, carrying oxygen and nutrients to nourish the heart tissue. The perfusion was initiated at a pressure resulted by placing the APEX hollow fiber membrane oxygenator with hardshell reservoir at 10 cm above the heart for 10 minutes. The perfusing pressure was then gradually increased by elevating height of the APEX hollow fiber membrane oxygenator with hardshell reservoir. Finally, the height of the APEX hollow fiber membrane oxygenator with hardshell reservoir was raised to 85 cm above the heart over a period of 10 minutes. The effluent gathered at right atrium from coronary sinus was drained from the second and third catheters having a balloon and a lumen. The initial 200 ml of the effluent containing a mixture of blood and the resuscitation fluid was discarded. The subsequent clear effluent was then drained continuously down into the second external reservoir. The effluent resuscitation fluid was re-circulated with a peristaltic pump to the APEX hollow fiber membrane oxygenator with hardshell reservoir where it was oxygenated with 95% O₂ and 5% CO₂. The first cardiac electric activity (QRS wave) was noticed at about 14 minutes after perfusion of the resuscitation fluid. Respiratory mechanical ventilation was resumed (tidal volume 15 ml/kg, rate 15/min, O₂ concentration 100%) at 14 minutes after perfusion of the resuscitation fluid. The heart rhythm reached about 40/minutes at 43 minutes after perfusion of the resuscitation fluid. The balloons in the superior, inferior vena cava, and the aorta were simultaneously deflated, and then the draining of the effluent in the right atrium and the perfusion of the resuscitation fluid in the aorta were stopped. The heart rhythm was about 60 beats/minutes at 60 minutes after perfusion of resuscitation fluid. O₂ concentration in respiratory mechanical ventilation was then changed from 100% to 30% for 10 minutes, and subsequently changed to atmospheric air. At 90 minutes after perfusion of the resuscitation fluid, the heart rhythm was about 61 beats/minutes and the blood pressure was 101/61 mm Hg. All catheters were then removed. Three hours after perfusion of the resuscitation fluid, the heart rhythm was about 63 beats/minutes and the blood pressure was 110/63 mm Hg. The ROSC was considered successful.

Conclusion: Unexpectedly, perfusing hearts with the resuscitation fluid as described above achieved ROSC after 30 minutes of cardiac arrest in pigs. By contrast, conventional CPR did not achieve ROSC after 30 minutes of cardiac arrest in pigs.

### Example 2

### Resuscitation Fluid Perfusion of the Whole Body to Treat Cardiac Arrest in a Rat Model

Preparation: Three male rats (the first rat weighing 300 grams, the second rat weighing 295 grams and third rat weighing 320 grams) were fasted overnight but provided water ad libitum before the resuscitating procedure. Cardiac arrest was induced in each rat as follow: 5% isoflurane was given for anesthetic induction. 2% isoflurane was given for anesthetic maintenance. The trachea was incubated and connected to a rodent ventilator (tidal volume 3 ml, rate 40/min, O₂ concentration 30%). The resuscitating procedure was conducted at room temperature 22-23° C. The left femoral vein and artery was catheterized via an inguinal cutdown. The venous catheter was used for administration of pharmacological agents. The arterial catheter was used for monitoring blood pressure. Cardiac rhythm was monitored by using a standard lead II electrocardiogram (EKG). Heparin was infused from femoral vein at 500 Units/kg. The pancuronium bromide 0.3 mg/kg was infused through femoral vein catheter to paralyze respiratory muscle's movement. Asphyxial cardiac arrest was induced by clamping of the endotracheal tube and stopping of the ventilator. Cardiac arrest was determined by isoelectric EKG.

Resuscitation with conventional CPR: At 45 minutes after cardiac arrest, the first rat was resuscitated with conventional CPR as follow: Mechanical ventilation was resumed (tidal volume 3 ml, rate 40/min, O₂ concentration 100%), and manual closed-chest CPR was initiated. Chest compressions were performed at a rate of 200 compressions/minute for 60 minutes. At 3 minutes after chest compressions, epinephrine was administered iv at 0.2 mg/kg. At 5 minutes after chest compressions, defibrillation was initiated with 10 j. At 10 minutes after chest compressions, epinephrine was administered iv at 0.4 mg/kg again. At 15 minutes after chest compressions, the defibrillation was given again with 20 j. As the ROSC was not achieved with 60 minutes of conventional CPR, the resuscitation attempt was given up.

Resuscitation with cell culture medium as the resuscitation fluid, whole body perfusion by two catheters and two external reservoirs: At 45 minutes after cardiac arrest, the second rat was resuscitated by perfusing its whole body with a resuscitation fluid as follow: Respiratory mechanical ventilation was resumed (tidal volume 3 ml, rate 40/min, O₂ concentration 100%). A first catheter (22 gauge, BD, catalog #384902), which was pre-filled with a resuscitation fluid was introduced from right femoral artery into the aortic arch such that the length of catheter inserted into the femoral artery was about 7 cm. The distal end of the lumen in the catheter was connected to a first external reservoir, and the distal end of the lumen was open to the aortic arch. A second catheter (20 gauge, BD, catalog #384902) was introduced from right femoral vein into right atrium via inferior vena cava. The proximal end of the lumen was open to right atrium, and the distal end of the lumen was connected to a second external reservoir. About 1,000 ml of a resuscitation fluid (i.e., a cell culture medium based formulation containing NaCl 6.4 g/L CaCl₂ 0.2 g/L, MgSO₄ 0.0977 g/L, KCl 0.4/L, NaHCO₃ 1.5 g/L, NaH₂PO₄.H₂O 0.125 g/L, L-Arginine.HCl 0.084 g/L, L-Cystine.2HCl 0.0626 g/L, L-Glutamine 0.584 g/L, Glycine 0.03 g/L, L-Histidine.HCl.H₂O 0.042 g/L, L-Isoleucine 0.105 g/L, L-Leucine 0.105 g/L, L-Lysine.HCl 0.146 g/L, L-Methionine 0.03 g/L, L-Phenylalanine 0.066 g/L, L-Serine 0.042 g/L, L-Threonine 0.095 g/L, L-Tryptophan 0.016 g/L, L-Tyrosine.2Na.2H₂O 0.10379 g/L, L-Valine 0.094 g/L, Choline Chloride 0.004 g/L, Folic Acid 0.004 g/L, myo-Inositol 0.0072 g/L, Nicotinamide 0.004 g/L, D-Pantothenic Acid 0.004 g/L, Pyridoxine.HCl 0.004 g/L, Riboflavin 0.0004 g/L, Thiamine.HCl 0.004 g/L, D-Glucose 1 g/L, Insulin 4 mg/L, and Albumin 2.5 g/L) was stored in the first external reservoir where it was oxygenated with 5% CO₂ and 95% O₂. The temperature of the resuscitation fluid was maintained at 22-23° C. An air bubble trap and a 0.22 μm sterile filter were installed between the first external reservoir and the first catheter. The resuscitation fluid from the first external reservoir was perfused into aortic arch with a peristaltic pump via the first catheter. The resuscitation fluid carried oxygen and nutrients to nourish the whole body of the rat. The perfusion rate was at 5 ml/minutes for the first 10 minutes. The perfusing rate was then increased to 10 ml/minutes for 10 minutes. The perfusing rate was subsequently increased to 20 ml/minutes for 10 minutes. Finally, the perfusing rate was set at 30 ml/minutes for 90 minutes. The effluent gathered at right atrium from coronary sinus was drained from the second catheter introduced from femoral vein. The initial 100 ml of effluent containing s mixture of blood and the resuscitation fluid was collected and centrifuged at 250 g for 10 min at 4° C. The blood cells were precipitated and saved. The supernatant was collected and concentrated by a tangential flow filtration apparatus with a membrane of low molecular weight cut off at 500 Daltons. The plasma was concentrated to about 20 ml with Tangential flow filtration apparatus. After this process, the plasma and blood cells was saved at 4° C. for later use. The clear effluent was drained continuously to the second external reservoir, and was transferred with a peristaltic pump to the first external reservoir where it was oxygenated. The first cardiac electric activity (QRS wave) was noticed at about 40 minutes after perfusion of the resuscitation fluid. The heart rhythm reached about 100 beats/minutes at 60 minutes after perfusion of the resuscitation fluid. The perfusion of the resuscitation fluid was then replaced by 25 ml of the second rat's own oxygenated blood (i.e., a mixture of plasma and blood cells was oxygenated with 95% O₂ and 5% CO₂ for 5 minutes). Upon the completion of blood perfusion, the first catheter in the aortic arch was closed. When blood appeared from the second catheter in the right atrium, drainage was stopped immediately. About 10 minutes later, the O₂ concentration in respiratory mechanical ventilation was changed from 100% to 30% for 10 minutes, then changed to atmospheric air. At 120 minutes after perfusion of the resuscitation fluid, the heart rhythm was about 160 beats/minutes and the blood pressure was 90/60 mm Hg. All catheters were then removed. Three hours after perfusion of the resuscitation fluid, the heart rhythm was about 240 beats/minutes and the blood pressure was 113/70 mm Hg. The ROSC was considered successful.

Resuscitation with the resuscitation fluid, whole body perfusion by two catheters and two external reservoirs: At 45 minutes after cardiac arrest, the third rat was resuscitated by perfusing its whole body with a resuscitation fluid as follow: Respiratory mechanical ventilation was resumed (tidal volume 3 ml, rate 40/min, O₂ concentration 100%). A first catheter (22 gauge, BD, catalog #384902), which was pre-filled with a resuscitation fluid was introduced from right femoral artery into the aortic arch such that the length of catheter inserted into the femoral artery was about 7 cm. The distal end of the lumen in the catheter was connected to a first external reservoir, and the distal end of the lumen was open to the aortic arch. A second catheter (20 gauge, BD, catalog #384902) was introduced from right femoral vein into right atrium via inferior vena cava. The proximal end of the lumen was open to right atrium, and the distal end of the lumen was connected to a second external reservoir. About 1,000 ml of a resuscitation fluid containing NaCl 118.5 mM, NaHCO₃ 25.0 mM, KCl 3 mM, MgSO₄ 2.5 mM, CaCl₂ 2.5 mM, Glucose 100 mg/dl, albumin 0.1 gram/dl, insulin 10 IU/L, heparin 10 U/L, and water was stored in the first external reservoir where it was oxygenated with 5% CO₂ and 95% O₂. The temperature of the resuscitation fluid was maintained at 22-23° C. An air bubble trap and a 0.22 μm sterile filter were installed between the first external reservoir and the first catheter. The resuscitation fluid from the first external reservoir was perfused into aortic arch with a peristaltic pump via the first catheter. The resuscitation fluid carried oxygen and nutrients to nourish the whole body of the rat. The perfusion rate was at 5 ml/minutes for the first 10 minutes. The perfusing rate was then increased to 10 ml/minutes for 10 minutes. The perfusing rate was subsequently increased to 20 ml/minutes for 10 minutes. Finally, the perfusing rate was set at 30 ml/minutes for 90 minutes. The effluent gathered at right atrium from coronary sinus was drained from the second catheter introduced from femoral vein. The initial 100 ml of effluent containing s mixture of blood and the resuscitation fluid was collected and centrifuged at 250 g for 10 min at 4° C. The blood cells were precipitated and saved. The supernatant was collected and concentrated by a tangential flow filtration apparatus with a membrane of low molecular weight cut off at 500 Daltons. The plasma was concentrated to about 20 ml with Tangential flow filtration apparatus. After this process, the plasma and blood cells was saved at 4° C. for later use. The clear effluent was drained continuously to the second external reservoir, and was transferred with a peristaltic pump to the first external reservoir where it was oxygenated. The first cardiac electric activity (QRS wave) was noticed at about 40 minutes after perfusion of the resuscitation fluid. The heart rhythm reached about 105 beats/minutes at 60 minutes after perfusion of the resuscitation fluid. The perfusion of the resuscitation fluid was then replaced by 25 ml of the second rat's own oxygenated blood (i.e., a mixture of plasma and blood cells was oxygenated with 95% O₂ and 5% CO₂ for 5 minutes). Upon the completion of blood perfusion, the first catheter in the aortic arch was closed. When blood appeared from the second catheter in the right atrium, drainage was stopped immediately. About 10 minutes later, the O₂ concentration in respiratory mechanical ventilation was changed from 100% to 30% for 10 minutes, then changed to atmospheric air. At 120 minutes after perfusion of the resuscitation fluid, the heart rhythm was about 154 beats/minutes and the blood pressure was 85/60 mm Hg. All catheters were then removed. Three hours after perfusion of the resuscitation fluid, the heart rhythm was about 250 beats/minutes and the blood pressure was 103/60 mm Hg. The ROSC was considered successful.

Conclusion: Unexpectedly, perfusing the whole body with the resuscitation fluid described above achieved ROSC after 45 minutes of cardiac arrest in rats. By contrast, conventional CPR did not achieve ROSC after 45 minutes of cardiac arrest in rats.

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations. Such equivalents are intended to be encompassed by the following claims.

What is claimed is:

1. A method of resuscitating a cardiac arrest patient, comprising the steps of:
A. introducing a first catheter having a lumen and a balloon into the aorta of the patient from a selected peripheral artery, inflating the balloon of the first catheter to block the aorta, and establishing fluid communication between heart coronary arteries and a first external reservoir via the lumen of the first catheter;
B. introducing a second catheter have a lumen and a balloon and a third catheter having a lumen and a balloon into the right heart of the patient from selected peripheral veins, inflating the balloons of the second and the third catheter to block superior vena cava and inferior vena cava, and establishing fluid communication between the right heart and a second external reservoir via the lumens of the second and third catheters;
C. perfusing the heart with a resuscitation fluid from the first external reservoir via the first catheter, wherein the resuscitation fluid comprises: Na⁺ 120-155 meq/L, Cl⁻ 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L and water, and the resuscitation fluid has an osmolality of 280-500 mOsm/L, a pH of 7-7.45, PO₂≧100 mm Hg, and PCO₂≦40 mm Hg;
D. draining an effluent of the resuscitation fluid into the second external reservoir from the right heart via the second and third catheters;
E. circulating the effluent of the resuscitation fluid from the second external reservoir into the first external reservoir where the resuscitation fluid is continuously oxygenated with CO₂ and atmosphere or O₂; and
F. deflating the balloons of the first, second and third catheters to resume blood circulation when the patient's heart beat on its own.

2. The method of claim 1, wherein the resuscitation fluid comprises NaCl 118.5 mM, NaHCO₃ 25.0 mM, KCl 4.7 mM, MgSO₄ 1.2 mM, glucose 11 mM, and CaCl₂ 2.5 mM.

3. The method of claim 1, wherein the resuscitation fluid comprises Na⁺ 130 mM, Cl⁻ 109 mM, K⁺ 4 mM, and Ca²⁺ 1.5 mM.

4. The method of claim 1, wherein the resuscitation fluid comprises 0.9 wt % of NaCl.

5. The method of claim 1, wherein the resuscitation fluid is a serum.

6. The method of claim 1, wherein the resuscitation fluid is a cell culture medium.

7. The method of claim 1, wherein the resuscitation fluid is continuously oxygenated with 5% CO₂ and 95% atmospheric air in step E.

8. The method of claim 1, wherein the resuscitation fluid is continuously oxygenated with 5% CO₂ and 95% O₂ in step E

9. The method of claim 1, wherein the resuscitation fluid is perfused into the aorta in step C at a pressure between 1-120 mm Hg.

10. The method of claim 1, wherein draining the effluent of the resuscitation fluid from the right heart in step D keeps central venous pressure below 8 mm Hg.

11. The method of claim 1, wherein CO₂ and O₂ are introduced into the resuscitation fluid in step E by a cardiopulmonary bypass oxygenator, and the first or second external reservoir is a cardiopulmonary bypass blood reservoir.

12. The method of claim 1, wherein the resuscitation fluid is perfused in step C at a temperature between 13° C. to 37° C.

13. A method of resuscitating a cardiac arrest patient, comprising the steps of:
A. introducing a first catheter having a lumen into the aorta of the patient from a selected peripheral artery to establish fluid communication between the aorta and a first external reservoir via the lumen of the first catheter;
B. introducing a second catheter having a lumen into the venous system to establish fluid communication between the venous system and a second external reservoir via the lumen of the second catheter;
C. perfusing the patient with a resuscitation fluid from the first external reservoir via the first catheter, wherein the resuscitation fluid comprises: Na⁺ 120-155 meq/L, Cl⁻ 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L and water, and the resuscitation fluid has an osmolality of 280-500 mOsm/L, a pH of 7-7.45, PO₂≧100 mm Hg, and PCO₂≦40 mm Hg;
D. draining blood and an effluent of the resuscitation fluid from the venous system into the second external reservoir via the lumen of the second catheter;
E. circulating the effluent of the resuscitation fluid from the second external reservoir into the first external reservoir where the resuscitation fluid is continuously oxygenated with O₂ or CO₂ and atmospheric air; and
F. infusing blood in the aorta to resume blood circulation when patient's heart beat on its own.

14. The method of claim 13, wherein the resuscitation fluid comprises NaCl 118.5 mM, NaHCO₃ 25.0 mM, KCl 4.7 mM, MgSO₄ 1.2 mM, glucose 11 mM, and CaCl₂ 2.5 mM.

15. The method of claim 13, wherein the resuscitation fluid comprises Na⁺ 130 mM, Cl⁻ 109 mM, K⁺ 4 mM, and Ca²⁺ 1.5 mM.

16. The method of claim 13, wherein the resuscitation fluid comprises 0.9 wt % of NaCl.

17. The method of claim 13, wherein the resuscitation fluid is a serum.

18. The method of claim 13, wherein the resuscitation fluid is a cell culture medium.

19. The method of claim 13, wherein the resuscitation fluid is continuously oxygenated with 5% CO₂ and 95% atmospheric air in step E.

20. The method of claim 13, wherein the resuscitation fluid is continuously oxygenated with 5% CO₂ and 95% O₂ in step E.

21. The method of claim 13, wherein the resuscitation fluid is perfused into the aorta in step C at a pressure between 1-120 mm Hg.

22. The method of claim 13, wherein draining the effluent of resuscitation fluid from the right heart in step D keeps central venous pressure below 8 mm Hg.

23. The method of claim 13, wherein the blood used in step F is patient's own blood collected in step D.

24. The method of claim 13, wherein the blood is processed by centrifugation and filtration before being infused into the aorta.

25. The method of claim 13, wherein CO₂ and O₂ are introduced into the resuscitation fluid in step E by a cardiopulmonary bypass oxygenator, and the first or second external reservoir is a cardiopulmonary bypass blood reservoir.

26. The method of claim 13, wherein the resuscitation fluid is perfused in step C at a temperature between 0.1° C. to 37° C.

27. A method of resuscitating a cardiac arrest patient, comprising the steps of:
A. introducing a first catheter having a lumen and a balloon into the aorta of the patient from a selected peripheral artery, and inflating the balloon to block the aorta;
B. introducing a second catheter having a lumen into the right heart of the patient from a selected peripheral vein;
C. perfusing the heart with blood via the lumen of the first catheter, and
D. withdrawing blood from the right heart via the lumen of the second catheter.

28. The method of claim 27, wherein the blood has PO₂≧100 mm Hg and PCO₂≦40 mm Hg.

29. The method of claim 27, wherein withdrawing blood from the right heart in step D keeps central venous pressure below 8 mm Hg.

30. The method of claim 27, wherein the heart is perfused with blood in step C at a pressure between 1-120 mm Hg.

31. The method of claim 27, wherein the blood perfused in step C has a temperature between 13° C. to 37° C.

32. A method of resuscitating a cardiac arrest patient, comprising:
infusing a resuscitation fluid into the arterial system, wherein the resuscitation fluid comprising: Na⁺ 120-155 meq/L, Cl⁻ 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L and water, and the resuscitation fluid having an osmolality of 280-500 mOsm/L, a pH of 7-7.45, PO₂≧100 mm Hg, and PCO₂≦40 mm Hg; and
draining an effluent of resuscitation fluid from the venous system.

33. The method of claim 32, wherein the resuscitation fluid is infused at a temperature between 0.1° C. to 37° C.

34. The method of claim 32, wherein the resuscitation fluid is perfused into the aorta in step C at a pressure between 1-120 mm Hg.

35. The method of claim 32, wherein the resuscitation fluid is a cell culture medium.

36. A method of providing life support during open heart surgery or aortic surgery of a patient when heart beat is stopped or aorta is cross-clamped, comprising:
perfusing the whole body of a patient with a resuscitation fluid, and
circulating the resuscitation fluid by a cardiopulmonary bypass equipment;
wherein the resuscitation fluid comprises: Na⁺ 120-155 meq/L, Cl⁻ 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L and water, and the resuscitation fluid has an osmolality of 280-500 mOsm/L, a pH of 7-7.45, PO₂≧100 mm Hg, and PCO₂≦40 mm Hg.

37. The method of claim 36, wherein the resuscitation fluid is infused at a temperature between 0.1° C. to 37° C.

38. The method of claim 36, wherein the resuscitation fluid is a cell culture medium.

39. A kit of resuscitating a cardiac arrest patient, comprising: at least a catheter having a lumen, at least a catheter having a balloon and a lumen, at least a reservoir, at least a pump, tubes for interconnection, a source of O₂ and CO₂, and a resuscitation fluid;
wherein the resuscitation fluid comprises: Na⁺ 120-155 meq/L, Cl⁻ 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L and water.

40. The kit of claim 39, wherein the resuscitation fluid comprises a cell culture medium.

41. A method of resuscitating a cardiac arrest patient, comprising:
perfusing the whole body of a patient with a resuscitation fluid, and
circulating the resuscitation fluid by a cardiopulmonary bypass equipment;
wherein the resuscitation fluid comprises: Na⁺ 120-155 meq/L, Cl⁻ 120-155 meq/L, K⁺ 0-5.0 meq/L, Ca²⁺ 0.1-3.0 meq/L, P 0-2 meq/L, Mg²⁺ 0.4-8 meq/L, HCO₃ 0-25 meq/L, Glucose 0-500 mg/dl and albumin 0-8 gram/dl, insulin 0-24 IU/L, heparin 0-10 U/L and water, and the resuscitation fluid has an osmolality of 280-500 mOsm/L, a pH of 7-7.45, PO₂≧100 mm Hg, and PCO₂≦40 mm Hg.

42. The method of claim 41, wherein the resuscitation fluid is perfused at a temperature between 0.1° C. to 37° C.

43. The method of claim 41, wherein the resuscitation fluid comprises a cell culture medium.

44. The method of claim 41, wherein the resuscitation fluid is perfused at a pressure between 1-120 mm Hg.
